(19) United States
(12) Patent Application Publication
(10) Pub. No.: US 2026/0194340 A1
(43) Pub. Date: Jul. 9, 2026

(54) INTERFEROMETRIC LASER SPECKLE CONTRAST IMAGING SYSTEM AND METHOD

(71) Applicants: The Medical College of Wisconsin, Inc., Milwaukee, WI (US); Coherence Engineering LLC, Durnham, NC (US)

(72) Inventors: Daniel Mark Lipinski, Milwaukee, WI (US); Al-Hafeez Dhalla, Durham, NC (US); Christian B. Viehland, Durham, NC (US)

(21) Appl. No.: 19/133,539

(22) PCT Filed: Dec. 1, 2023
(86) PCT No.: PCT/US2023/082063

§ 371 Date: May 28, 2025

Related U.S. Application Data
(60) Provisional application No. 63/430,080, filed on Dec. 5, 2022.

(51) Int. Cl. G01B 9/02; A61B 5/00; G01B 9/02091

(52) U.S. Cl. CPC G01B 9/02096; A61B 5/0066; G01B 9/02091

(57) ABSTRACT

An interferometric laser speckle contrast optical system configured to limit depth of field in order to image only a thin curved object layer of a chosen object while distinguishing such layer from the surrounding curved layers that substantially are not being imaged. The system utilizes a novel 5F-optical arrangement in a sample arm of the constituent interferometer to substantially match the curvature of the surface of illumination with that of the targeted object layer, thereby increasing the spatial resolution and reducing the signal-to-noise ratio of the imaging process. When used as part of an ophthalmoscope, the system for hemodynamic alterations to be measured with substantially capillary level resolution and to establish normative baseline values for retinal neurovascular function.

### CROSS-REFERENCE TO RELATED APPLICATIONS

This International Patent Application claims priority from and benefit of the U.S. Provisional Patent Application No. 63/430,080 filed on Dec. 5, 2022, the disclosure of which is incorporated herein by reference.

### STATEMENT CONCERNING FEDERALLY SPONSORED RESEARCH AND DEVELOPMENT

This project was funded under grant R01EY027767 by the National Eye Institute. The government has certain rights in the invention.

### TECHNICAL FIELD

The present invention relates to laser speckle imaging and, more particularly, to a methodology of scanning interferometric optical imaging of a target with restricted longitudinal spatial resolution to purposefully avoid collecting optical information provided by the object space neighboring the target while, at the same time, dynamically—not statically—spatially conforming or matching the spatial distribution of illumination delivered to the object through the optical system to the object surface being imaged, to increase the useful field-of-view of the imaging apparatus.

### RELATED ART

Laser speckle contrast imaging (LSCI)—sometimes referred to as laser speckle imaging (LSI)—is an imaging modality the operation of which turns on the analysis of the blurring effect of the speckle pattern. The operation of LSCI involves, generally, a wide-field illumination of a rough object surface with light generated by a coherent light source. Then, with the use of photodetectors (such as CCD cameras or CMOS sensors, for example) imaging is caried out of the resulting laser speckle pattern caused by the interference of coherent light. (In a specific case of biomedical use, the coherent light is typically in the red or near-infrared spectral region to ensure higher penetration depth of light into the target tissue.)

In this regard, LSCI is a promising, but under-utilized, non-invasive, and non-contact imaging technique capable of generating wide-field maps of blood flow for quantifying retinal hemodynamics, without requiring exogenous contrast. Just like in any other application of the LSCI, illumination of a retinal blood vessel, for example, with light generated by a coherent light source results in the formation of a random ‘speckle pattern’, where the intensity of each pixel results from the coherent addition of backscattered light with different optical path lengths. Movement within the field-of-view, FOV (e.g., moving blood cells) causes temporal and spatial fluctuations in the speckle pattern such that the rate, at which the intensity of each pixel changes over time, is characterized by the decorrelation time of the speckle autocorrelation function. When this dynamic speckle pattern is recorded over a finite integration time set by the duration of the camera's exposure, the longer integration time relative to the decorrelation time results in a speckle blurring effect (the extent of which is referred to as speckle contrast, K). Speckle contrast K is typically quantified as the ratio of the standard deviation of time-integrated speckle intensities to their mean intensity (Eq. 1) within a small spatial window of 5×5 or 7×7 pixels, for example, (referred herein as ‘spatial processing’) for high temporal resolution, or at the same pixel position across time (referred herein as ‘temporal processing’) for high spatial resolution.

$$K = \frac{σ}{〈 I 〉} \qquad ( 1 )$$

Situations in which the existing LSCI devices are not able to resolve adequately particle/element motion in objects generally include circumstances when the longitudinal (axial, measured along the interrogating beam of light) extent of an object is substantially small and light returned to the optical imaging system by surrounding portions of space substantially washes out the otherwise useful signal information received from the object. In a specific example of a biomedical use, existing LSCI devices for human use are unable to resolve and assess hemodynamic changes in the microvasculature. (See, for example, Patel, D. D. et al., “Development of a Preclinical Laser Speckle Contrast Imaging Instrument for Assessing Systemic and Retinal Vascular Function in Small Rodents”, in Translational vision science & technology 10, 19 2021; Cho, K. A., et al. “Portable, non-invasive video imaging of retinal blood flow dynamics”; in Scientific reports, 10, 20236, 2020: Feng, X. et al. “Functional imaging of human retina using integrated multispectral and laser speckle contrast imaging”, in J Biophotonics, e202100285, 2021.) This is a critical technology gap that prevents LSCI from being used as an effective diagnostic tool, wherein vascular dysfunction in Alzheimer's disease (AD; and co-morbid conditions) primarily affects smaller vessels, such as capillaries, initially or exclusively.

### SUMMARY OF THE INVENTION

Embodiments of the invention provide a system that includes an optical imaging apparatus. The optical imaging apparatus contains an optical interferometer having a sample arm and a reference arm, and an optical relay sub-system in the sample arm. Such the optical relay sub-system is configured to include first and second lenses (axially separated by a distance exceeding a sum of focal lengths of the first and second lenses—in one case, necessarily exceeding) and to form a spatially-curved image surface that has a corresponding curvature that is variable as a function of the distance. In at least one case, the system has a full field of view of at least 15 degrees and/or of at least 30 degrees and/or each of the first and second lenses is structured as an optical doublet (which lenses, in one specific implementation, may be structured as substantially identical optical doublets). Substantially in every implementation, the system may be configured as an ophthalmoscope and may additionally include a source of light (with a coherence length that is substantially within a range from about 200 microns to about 450 nm). Alternatively or in addition—and substantially in every embodiment—the system may include a source of light and be configured to irradiate an object with light formed at the output of such source of light apparatus while registering, at an optical detector of the system, a laser speckle representing the object. (At least in one case, such source of light may be configured to interchangeably form or produce light either as a first light or as a second light such that the first and second lights have corresponding degrees of temporal coherence that are different from one another.) Alternatively or in addition—and substantively in every embodiment—the system may include a multi-port optical device, a source of light that contains a light emitter (such as, for example, a laser diode) that is optically coupled to a first port of the multi-port optical device, an optical reflector optically coupled to a second port of the multi-port optical device, and an optical switch optically coupled to both the second port and a third port of the multi-port optical device. Alternatively or in addition—and substantially in every implementation—the system may include a source of light that is characterized by or includes an input (defined by a light emitter having a first spectral bandwidth), an intermediate portion of the source of light, and an output (defined by an optical switch that is optically separated from the input by the intermediate portion). In such a case, the intermediate portion is configured to deliver light from the input to the output along first and second optical paths (here, the first optical path is configured to include an optical reflector and to deliver from the light emitter to the output a first light having a bandwidth that is substantially equal to the first bandwidth while the second optical path is configured to deliver from the light emitter to the output a second light having a spectral bandwidth substantially equal to a spectral bandwidth of the optical reflector). In more than one of the above-identified embodiments, the source of light may be optionally configured to deliver towards the optical interferometer an optical output that is formed—interchangeably—in either a first optical output mode or in a second optical output mode (here, a spectral bandwidth of the optical output in the first output mode is at least ten times narrower than that in the second output mode). Alternatively or in addition—and substantially in every implementation—the system may include an optical circulator, an optical reflector, an optical amplifier, and an optical switch that is optically separated from the optical circulator by each of the optical circulator, the optical reflector, and the optical amplifier. Optionally, every implementation of the system may be complemented by a programmable data-processing electronic circuitry (or, simply put, a processor) that is operably connected with an optical detector of the system and tangible non-transitory storage medium. Such storage medium contains program code thereon, which program code is configured to determine an index of motion (at or in an object irradiated with light that has traversed the optical interferometer) based at least in part on a speckle contrast characteristic of at least one optical image of the object formed at the optical detector. Optionally, such object may be represented by a layer of retina or a layer of choroid of an eye (in which case the program code is configured to determine a blood flow rate in a reference blood-vessel of the layer of retina or the layer of choroid).

Embodiments of the invention additionally provide method that, with the use of substantially every embodiment of the system alluded to above, performs at least the following steps: (a) a step of dynamically matching a first spatial curvature of an arcuate surface of illumination (formed by illuminating light propagating from a source of light of the system through an illumination sub-system) with (or spatially confirming such first spatial curvature to) a second spatial curvature of a spatially curved object layer of a material—or, put differently, spatially curved layer of an object. (Here, the illumination sub-system includes a sample arm of an optical interferometer.); (b) a step of irradiating the spatially curved object layer with the illuminating light by substantially spatially superimposing the arcuate surface of illumination with the spatially curved object layer; and (c) forming—at an optical detector of a light collecting sub-system—an optical image of the curved object layer by spatially overlapping sample light (which has been returned from said spatially curved object layer irradiated with the illuminating light) with a portion of the illuminating light that has been delayed with respect to the sample light. In at least one embodiment, the method step of dynamically matching may be configured to include substantially matching the first spatial curvature with the second spatial curvature over at least a 15-degree full field of view (FOV) of the illumination sub-system, and/or over at least a 20-degree full FOV of the illumination system, and/or over at least a 30-degree full FOV of the illumination sub-system. Optionally, the method is characterized by at least one of the following: (i) the step of dynamically matching is carried out substantially simultaneously with the step of irradiating, (ii) the illuminating surface is substantially optically-conjugate with the source of light, (iii) the step of irradiating includes irradiating the spatially curved object layer with the illuminating light that has a coherence length that necessarily does not exceed a thickness of the spatially curved object layer of a material and/or that is substantially equal to such thickness. Notably, substantially in every implementation of the method, the spatially curved object layer of a material may include a spatially curved stack of multiple material layers. Optionally, a step of dynamically matching of at least one implementation of the method may include axially repositioning a first lens of a multi-lens optical relay sub-system that is contained within the sample arm of the optical interferometer. Alternatively or in addition, and substantially in every implementation of the method, the step of forming an optical image may be configured to include forming an interferometric image of only the spatially curved object layer of a material (and not of another portion of an object space that is adjacent to the spatially curved object layer of a material) by optically interfering the sample light with the portion of illuminating light at the optical detector. Substantially every embodiment of the method may optionally include a step of generating first and second images of the spatially curved object layer of a material—which images respectively represent such curved layer at different depths thereof—by changing a delay between the sample light and the portion of illuminating light. (In one specific implementation of the method containing such step of generating first and second images, the spatially curved object layer of a material may be chosen to includes only one—and not more—structural layer of multiple structural layers located at a back of an eye, in which case the step of generating is configured to include depth-wise imaging of such only one structural layer while not imaging another of the multiple structural layers.) The step of forming an optical image may include forming an optical image that simultaneously representing a microvasculature of the retina of an eye over at least a 15-degree full field of view, and/or over at least a 20-degree full field of view, and/or over at least a 30-degree full field of view. Alternatively or in addition—and substantially in every implementation—the method may additionally include a step of forming the illuminating light interchangeably (as a first illuminating light or as a second illuminating light) by transmitting light that has been emitted by a light emitter through an optical switch that optically separates the light emitter from the optical interferometer (here, the first and second illuminating lights are characterized by respectively corresponding first and second spectral bandwidths that are different from one another—in one specific case, by at least an order of magnitude in order to ensure the ability to illuminate/irradiate the object with different lights the degrees of coherence of which are substantially incomparable). When the illuminating light is so formed interchangeably, the method may additionally include forming the second illuminating light at least in part by spatially overlapping (i) a first portion of the light that has been emitted by the light emitter and that has been reflected at an optical reflector and (ii) a second portion of the light that has been emitted by the light emitter. (Optionally, both the first and second portions of the light that has been emitted by the light emitter may be additionally amplified after such first and second portions are spatially combined by overlapping.) The process of spatially overlapping may include transmitting both the first portion and the second portion through the same port of a multi-port optical device that is optically separated from the optical switch by the optical reflector. More than one embodiment of the method may further employ a step of delivering light that has been emitted by the light emitter to the optical switch along first and second spatially distinct from one another optical paths (the first optical path traversing an optical circulator and a fiber Bragg grating and the second optical path traversing the optical circulator and an optical amplifier). Alternatively or in addition—and substantially in every embodiment—the method may include a step performed with the use of a programmable data-processing electronic circuitry (or processor, for short) operably connected with the optical detector: such step is the step of determining an index of motion occurring in or at the spatially curved object layer, and the determining is carried out based at least in part on a speckle contrast characteristic of the optical image of the spatially curved object layer. Optionally—when such determining is taking place—the method may be complemented with generating a visually-perceivable output representing changes in dynamics of the motion over a predetermined period of time based at least on the results of the determining. Understandably, if and when the curved object layer of material is a layer of a biological tissue, the index of motion may represent a parameter of a blood flow in such biological tissue. For example, when the curved object layer of material is a layer of retina or a layer of choroid of an eye, the step of determining an index of motion may include determining a blood flow rate in a reference blood-vessel at or within such curved object layer. (In this case, the method may be additionally complemented with generating a visually-perceivable output representing quantified changes in retinal hemodynamics over a predetermined period of time based at least on the determining.)

Embodiments of the invention additionally include a computer program product incorporating program code(s) that embody various implementations of the method alluded to above.

### BRIEF DESCRIPTION OF THE DRAWINGS

The invention will be more fully understood by referring to the following Detailed Description of Specific Embodiments in conjunction with the Drawings, of which:

FIG. 1 is a schematic diagram of an eyeball.

FIG. 2 schematically illustrates the optical imaging system according to a generalized embodiment of the invention the use of which illustrates the workability of the proposed methodology.

FIG. 3 shows the optical layout of the conventional 4F-optical relay in comparison with the optical layout of an embodiment of the optical relay shown in FIG. 4 and employed in the sample arm of the interferometer of the embodiment of the optical imaging system of the invention.

FIGS. 5A, 5B illustrate various field curvatures, as discussed.

FIGS. 6A, 6B provide spot diagrams characterizing a conventional 4F-type optical relay and that configured according to the embodiment of the invention, comparing the two at different field angles.

FIG. 7 Plot of optical-path-length difference (OPLD) of light propagated through the embodiment of optical sub-system 110, showing less than ±0.1 mm roundtrip OPLD across an approximately 15° half-FOV.

FIG. 8A illustrates a specific version of the generalized embodiment of the optical system of the invention shown in FIG. 4, which is characterized by the same performance characteristics as those discussed in reference of the generalized embodiment of FIG. 4.

FIGS. 8B, 8C, and 8D illustrate portions of the optical train of the embodiment depicted in FIG. 8A.

FIG. 9 depicts a modeled reference wavefront at a surface of the CMOS of the specific embodiment of FIG. 8A, demonstrating a wavefront error that is smaller than 0.16λ peak-to-valley.

FIG. 10 provides an illustration of the path of illumination of the curved layer of the object (in this non-limiting example chosen to be a curved layer of an eye)—on the left, and an illustration of the path of collection of light in reflection from (or scattering by) such curved layer of the object—on the right. Both illustrations shown light beams pivoting through the center of curvature of the curved layer of the object.

FIG. 11 presents both the plots representing the modulated transfer function (MTF; top portion of the drawing) and spot diagrams (bottom) depicting diffraction limited performance of the embodiment of FIG. 8A while imaging the retina of an eye to CMOS.

FIG. 12 is a schematic representation of one specific embodiment of a source of light, with which the embodiment of the optical system of FIGS. 4 and/or 8A may be complemented in practice.

FIG. 13 is a plot illustrating a typical optical spectrum of the light output produced by the embodiment of the source of light of FIG. 12.

Generally, the sizes and relative scales of elements in Drawings may be set to be different from actual ones to appropriately facilitate simplicity, clarity, and understanding of the Drawings. For the same reason, not all elements present in one Drawing may necessarily be shown in another. While specific embodiments are illustrated in the figures with the understanding that the disclosure is intended to be illustrative, these specific embodiments are not intended to limit the scope of invention implementations of which are described and illustrated herein.

### DETAILED DESCRIPTION

In accordance with the idea of the present invention, embodiments of an LSCI apparatus and related methods are realized that employ interferometric optical imaging to purposefully and necessarily limit the depth of field to lengths substantially comparable with the spatial extent of the target object being imaged along an axis of the interrogating beam of light. Implementations of the idea of the invention results in determination of parameter(s) of a motion occurring at or in the object only, without accounting for optical information received from a portion of space next to the target object, thereby effectively increasing the signal-to-noise ratio of the measurement. In one specific and non-limiting case—when applied to a living tissue, such as the retina, for example—embodiments of the proposed LSCI methodology can be used to make quantitative measurements of hemodynamics, including any alterations in blood flow or vascular function, on a very short spatial scale. (As the retina is an extension of the central nervous system and undergoes neuronal degeneration in AD, imaging of the retinal vasculature may provide a promising alternative approach for assessing neurovascular health in patients with dementia. Critically, as the retinal vasculature can be visualized directly and non-invasively through the pupil, it is expected that even minor structural or functional abnormalities may be detectable at a pre-clinical disease stage, thereby providing an unparalleled opportunity to diagnose AD early, assess progression over time, or monitor the effects of any therapeutic intervention.) However, as the skilled artisan will readily appreciates, the use of and/or reference to any portion of an eye in the discussion below is but a specific example, with understanding that the proposed apparatus and methodology are and should be applicable to optical imaging in general.

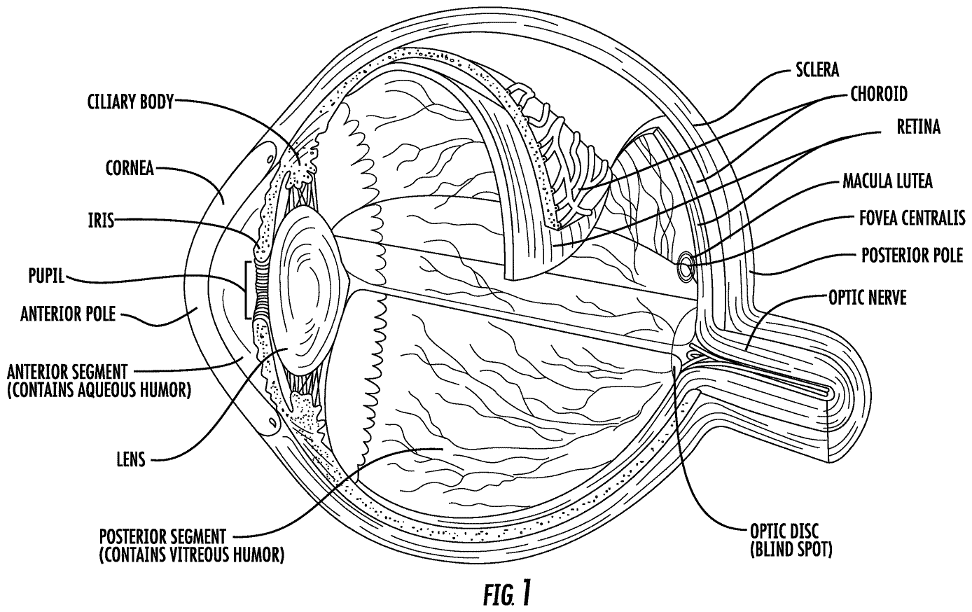

The need in configuring embodiments of the invention to intentionally limit the depth of field—in start contradistinction with existing LSCI systems, as well as most full-field OCT systems—stems from the realization that the multi-layered nature of various objects (and, in the specific non-limiting example, the back portion of the eye—consider, for example, the schematic representation depicted in FIG. 1, where the material layers of choroid, sclera, and retina at the back of the eye are expressly shown) inevitably limits the ability of a conventional imaging system to have the signal-to-noise ratio (SNR) above a certain threshold level. (In a chosen specific non-limiting example of imaging an eye—when imaging, for example, vasculature of only one of choroid and retina and not another.) Indeed, the optical interferometric signal arriving from the neighboring layer (in the chosen example—the other of the choroid and retina) necessarily reduces the practically achievable SNR, thereby at least complicating—if leaving possible at all—the measurement of hemodynamic alterations to be measured with capillary level resolution.

The formation of the desired and intended shallow depth of field, according to the idea of the invention, is carried out by intentionally reducing and/or limiting the coherence length of light interrogating the sample/object to geometrical extent comparable with or even shorter than the thickness (axial extent) of the specific target object (thereby making such chosen coherence length, more generally, object dependent).

Moreover, embodiments of the invention are configured to vary the axial position of the region of object space imaged within the intentionally limited shallow depth of field by a scanning motion that is effectuated, according to the idea of the invention, via tuning a portion of optical interferometer that is part of the optical imaging system of an embodiment.

Furthermore—and in stark and advantageous contradistinction to currently available LSCI methodologies—the embodiments of the proposed LSCI apparatus are configured such as to take into account the spatial curvature of a target object (in the case of imaging the vasculature of the choroid and/or retina—the spatial curvature of these layers of the eye structure). This practical advantage provided by the discussed embodiments simply cannot be overestimated: in conventional designs of an LSCI system, where the imaging plane of the system is substantially flat, the natural curvature of the imaged object necessarily severely limits the field-of-view (FOV) of the LSCI since only the small area of the curved object (in one case—retina, choroid) that subtends central few degrees of angular space would be in the correct imaging plane. In stark contradistinction with what is available in related art, an embodiment of the invention is configured to dynamically adjust (that is, vary) the curvature of the image surface of the optical imaging system of the embodiment without changing the set of the optical elements or components that such embodiment includes.

Overall, as the skilled artisan will appreciate from this disclosure, the problem of inability of the existing LSCI methodologies to simultaneously, in a snap-shot, produce an image of a spatially curved object configured as a layer of material with limited thickness and spatially-resolve different depths of such object on an image-by-image basis (while, at the same time, negating the optical information arriving from portions of the object space surrounding the target object) is solved by devising an optical interferometer based scanning LSCI apparatus configured to image the target object in light having coherence length substantially equal to or shorter than the axial extent of the object while matching (that is, making substantially congruent) the curvature of the surface of illumination, produced by the apparatus, with that of the object.

It is appreciated that while the discussion of applications of the embodiments of the proposed methodology presented below includes discussing the imaging of the visual system of a human, such application is but a specific example chosen due to the availability of multiple spatially curved layers in such visual system. The skilled person will clearly understand that the use of embodiments of the proposed methodology are those utilizing non-animate objects as much as animate objects.

FIG. 1 provides a diagram of one specific target object—an eyeball, with identification some of the objects of interest—here, represented by the spatially curved layers of the choroid and/or retina of the eye.

### Example 1

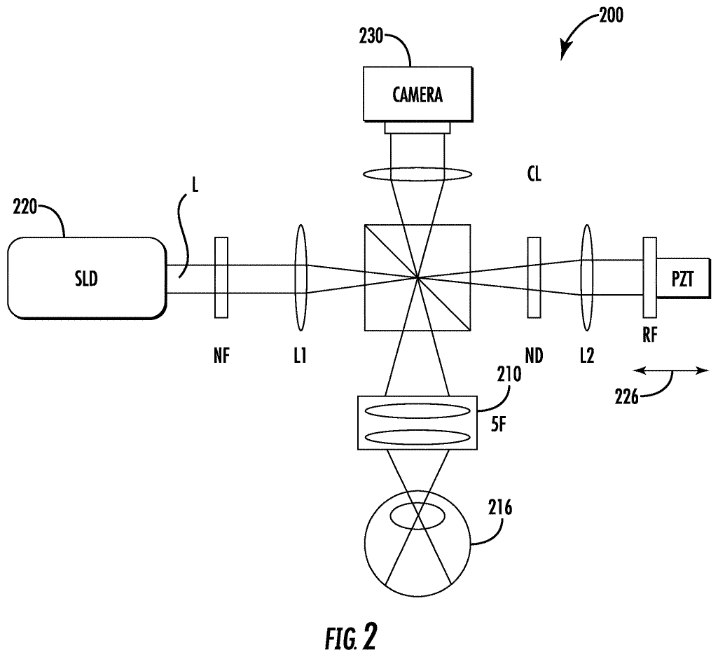

FIG. 2 illustrates a generalized schematic of embodiment 200 of the laser-speckle-imaging apparatus of the invention, which incorporates a custom-configured optics 210 into a sample arm of a free-space interferometer (shown in this specific case as a Michelson interferometer. When the target object to be imaged is a portion of the eye 216, the embodiment may be referred to as an interferometric laser speckle contrast ophthahnometer—or ILSCO. The optics 210 is judiciously configured to produce a curved imaging surface (subtending, in one case, at least a 15-degree—and preferably a 30-degree FOV) with a shallow, object-dependent depth of field limited to the axial extent of the object (about 250 microns to about 450 microns, in the case of the retina and/or the choroid). The object-dependent imaging fieldt can be centered on the target object (in the chosen example—either the neural retina or choroid) through adjustment of the reference arm of the apparatus 200. The reference arm is shown to include some optical elements (ND, neutral density filter; L2—optical lens; RF—a reflector at least axially driven by the micropositioner PZT—a piezo as shown). The apparatus 200 may additionally include another amplitude and/or spectral filter F disposed between the source of light 220 (generally including a light emitter and optionally additional constituent optical components required to appropriately configure light delivered to the interferometer; in this specific example shown simply as a superluminescent diode, SLD) configured to generate light L (at a wavelength of about 780 nm) with the desired low coherence length. Light L, after being substantially collimated with the collimating optics (not shown), is further directed through the lens L1 towards the beam splitter BS, and an auxiliary collimating lens CL through which the spatially-overlapped portions of light from the reference arm and the sample arm of the interferometric apparatus are delivered to the optical detection system 230.

In comparison with most full-field OCT systems or conventional LSCI systems of related art (which utilize light with coherence length on the order of at least a few microns), the choice of the optical source interchangeably referred to herein as a source of light 220 or a combination of the optical source 220 with the filter(s) F (for example, a notch filter) is such as to produce light with judiciously limited coherence length that is long enough to span the substantially all thickness of the retina (of about 250 microns to about 350 microns, on average), but short enough for the apparatus 200 to interferometrically reject light reflected from the choroid (with a typical thickness of about 200 microns to about 450 microns) or vice versa. Since the coherence length relates to a Fourier transform of the spectrum of light delivered to the target object 216, in the embodiment of this example the value of the desired coherence length can be chosen with the use of custom fabricated optical bandpass filters applied to light emanating from the narrowband SLD 226. A skilled person will readily appreciate that the intentionally limited coherence length of light used for imaging of the spatially-curved object 216 produces, in operation, the axial sectioning of the object in that in different LSCI images acquired with the optical detection system 230 with different lengths (as indicated by the arrow 226) of the reference arm of the apparatus 200 different sub-layers of the object 216 (or, different portions of the object located at different depths) will be imaged.

An additional operational feature of the embodiment 200—the need in which is not accounted for or even anticipated by the LSCI systems of related art—is that the apparatus 200 is configured to form the spatially curved imaging surface (or surface of illumination, which during the imaging is overlapped with the target object). Notably and referring to the specific example of imaging the back portion of the eye: while it is possible, in principle, to carefully select and arrange constituent optics to match (that is, to make substantially congruent with) a singular retinal curvature, natural variation in human anatomy necessarily leads to variations of a focal length of the natural lens of the eye, refractive indices, geometry of the eye, and curvature of the choroid/retina from subject to subject. Therefore, a static solution remains substantially impractical.

To address the need in a dynamic, in real time, change in the curvature of the surface of illumination the optics 210 is configured to changeably match the curvature of the retina by translating a single constituent lens of the optics 210 (which may be controlled by way of an adjustment knob by the operator or via a piezo-based repositioning, in one implementation) and/or a working distance (that is, the separation between the closest to the target object lend of the optical 210 and the target object).

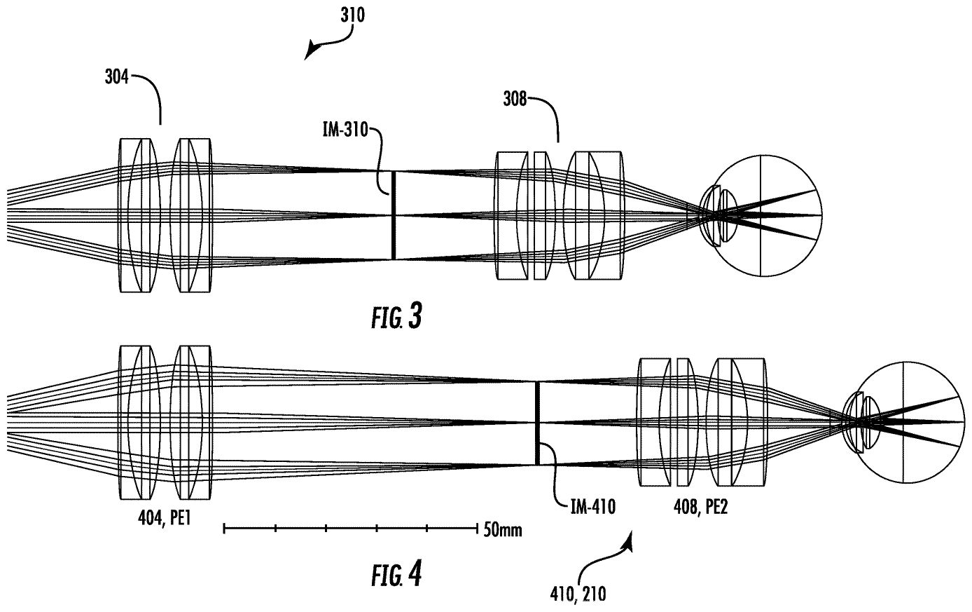

The idea of the proposed solution is illustrated in reference to FIGS. 3 and 4. FIG. 3 illustrates schematically an embodiment of the conventional so-called 4F optical system (essentially, a telescope with finite conjugates located one focal distance to the left of the objective lens 304 and one focal distance to the right of the collecting lens 308, respectively) that contains a combination of two lenses performing a cascade of Fourier transformations of light passing through such combination. FIG. 4 shows an embodiment of the optical system 410 (representing the optic 210 of FIG. 2), the operational length of which, in comparison and contradistinction with related art, is about 5F.

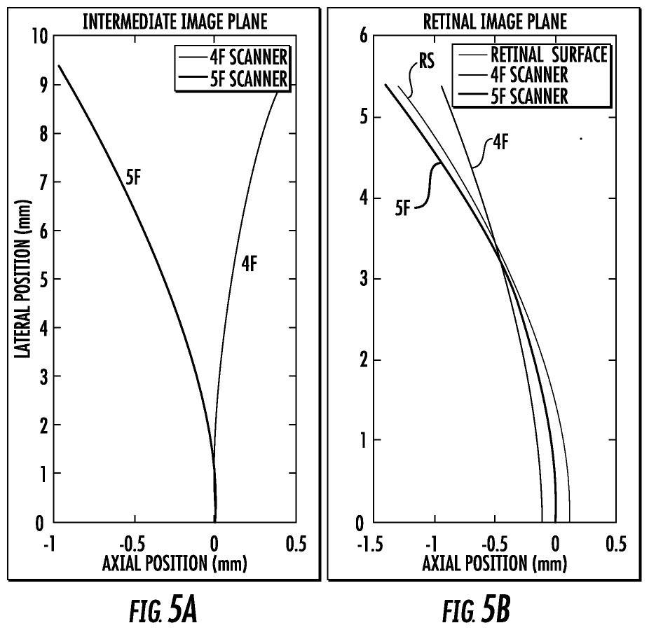

In the conventional 4F design of the optical system 310 of FIG. 3, the intermediate image plane IM-310 is devised to be as close to a telecentric position as possible, while the substantially 5F design of the embodiment of FIG. 4 the field curvature is intentionally induced in the intermediate image plane IM-410 (FIG. 5A). Once imaged onto the target object (for example, a curved retinal layer of the eye, RD of FIG. 5B), this additional intentionally formed and absent in operation of the conventional 4F-optical system field curvature creates an illumination surface that better matches the curvature of the retina, thereby minimizing defocus across the entire FOV (FIG. 5B).

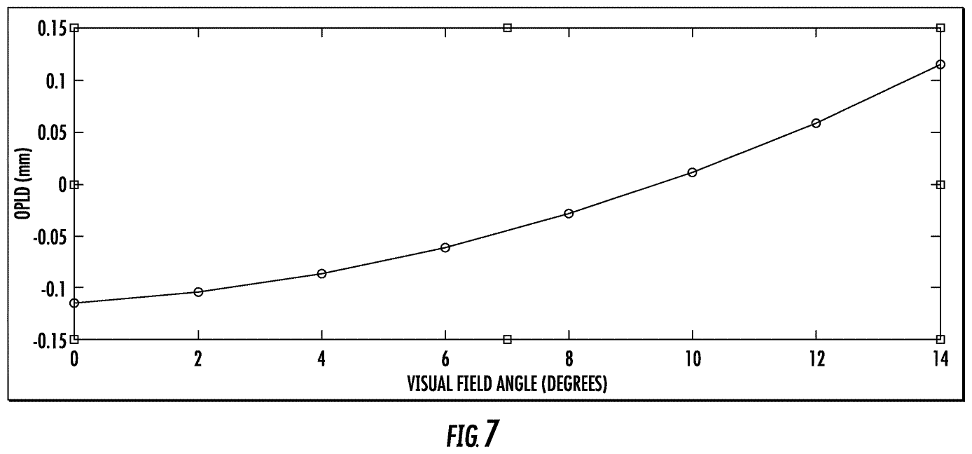

Referring again to FIGS. 2 and 4, in one implementation, the constituent lenses 404, 408 of the embodiment 410, 210 were custom Plossl eyepieces separated by a distance larger than larger than the sum of their focal lengths. When designed appropriately, this arrangement produces a curved image plane on the back of the eye, and also minimizes the optical pathlength difference (OPLD) across the half-FOV—see FIG. 7, which is critical to the ophthalmological application of the apparatus 100. The curvature of the back focal plane and OPLD surface can be adjusted by modifying the spacing between PE1 and PE2 and refocusing the camera 230 (with the working distance remaining constant).

FIGS. 5A, 5B are plots illustrating empirically and/or theoretically determined curvatures of the intermediate image planes/surfaces IM-310, IM-410 and the surfaces of illumination (final image surfaces) SI-224, SI-228 for the conventional 4F-type optical system of FIG. 3 employed in the embodiment of FIG. 2 and the embodiment of FIG. 4 employed in the embodiment of FIG. 2. The slight negative curvature of the spatial light distribution produced by the conventional system of FIG. 3 in the intermediate image plane IM-210 resulted at least from the lens aberrations.

In practice, both systems of FIGS. 3, 4 were implemented with the same commercially available lenses (Edmund Optics Inc., Barrington. NJ and Thorlabs, Newton, NJ), and were optimized using ray tracing software (Radiant Zemax LLC, Redmond, WA) and the Goncharov and Dainty eye model, a well-established adult eye model for non-wide field systems (see A. V. Goncharov and C. Dainty, “Wide-field schematic eye models with gradient-index lens,” J. Opt. Soc. Am. A 24(8), 2157-2174, 2007; the disclosure of which is incorporated herein by reference). Refractive error was modeled by modifying the axial length of the eye model (see A. Dubra and Y. Sulai, “Reflective afocal broadband adaptive optics scanning ophthalmoscope,” Biomed. Opt. Express 2(6), 1757-1768, 2011; incorporated herein by reference).

The design of the embodiment 410 of the system 210 (FIG. 4) was optimized such that the Strehl ratio remained above 0.7 over the 30° FOV and a range of corrected refractive errors. For both the conventional 4F system of FIG. 3 and the novel system 410, 210 of FIG. 4 translating the corresponding collimating lens (308, 408, respectively) changes the amount of field curvature correction.

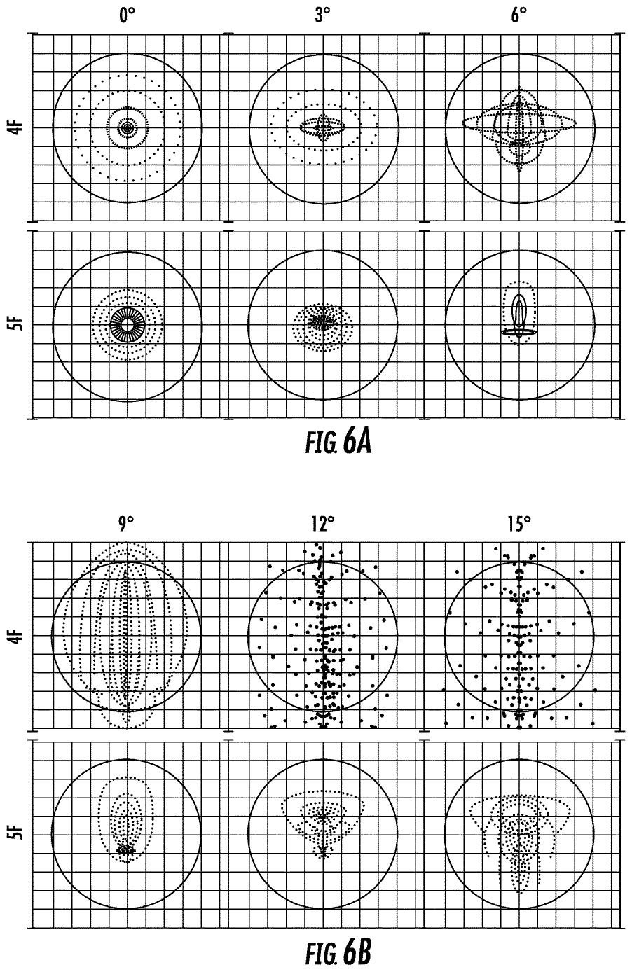

While both systems 310, 410 had the same diffraction limited resolution (12.0 μm Airy radius), the substantially 5F design 410 of the system 210 produced diffraction limited spots over a ~30° field of view (full angle, limited by clipping on a lens aperture) while the 4F design of FIG. 3 produced diffraction limited spots over only a ~16° degree field of view (see FIGS. 6A, 6B).

The embodiment 410, 210 of the optical system (placed in the sample arm of the interferometer of the apparatus 100) was configured according to the idea of the present invention to dynamically correct for a baseline radius of curvature of the object being imaged (for example, the retina or the choroid of the eye) of 12 mm, with dynamic adjustment of the working distance, for example, between about 11 mm and about 14 mm (thereby covering the typical range of curvatures of adult human retina), while achieving a spatial resolution of 8 microns (Airy radius) over at least a 15 degree (and in one specific related embodiment—a 30 degree) full FOV.

A skilled person having an advantage of this disclosure now readily appreciates that an embodiment of the invention provides methodology for rejecting the out-of-target-plane signals with the use of temporal coherence gating in which light emitted from a coherent light source (e.g. a laser diode) traverses the sample path and the reference path and, upon recombination, produces the interference signal if the optical pathlengths of the sample path and reference path match (within a particular length known as the coherence length). By shortening the coherence length to that substantially comparable with or smaller than the axial extent of the object, and using custom optical relay incorporated into an optical interferometer, an embodiment of the invention produces a curved imaging surface (with at least a 15 degree full FOV or in a related embodiment—at least a 20 degree full FOV, or even over at least a 30 degree full FOV) and a shallow depth of field (between about 250 and about 500 microns, depending on the choice of the coherence length of light as measured within the medium of the target object/sample thereby considering the group refractive index of the sample medium) sufficient for LSCI-based imaging of the motion of the elements of the curved object while substantially rejecting light scattered by the surrounding volumes of the object space and returned to the optical imaging system. In one specific implementation, this enables detection of flow information from the retinal microvasculature only, rejecting light reflecting from the underlying choroid—or vice versa. Accordingly, implementation of the idea of the invention manifests in a system containing an optical apparatus that includes (i) an optical interferometer having a sample arm and a reference arm; and (ii) an optical relay system in the sample arm, where the optical relay system is configured a) to include first and second lenses axially separated by a distance exceeding a sum of focal lengths of the first and second lenses, and b) to form a spatially-curved image surface having a curvature that is necessarily variable as a function of said distance and the working distance of the imaging apparatus.

For LSCI imaging of the human visual system, one does not have to be concerned with axial resolution, but rather only axial sectioning. That is, optionally, during the operation of an embodiment of the apparatus discussed above while imaging the back portion of the eye only the rejection of the optical signal returned from the choroid signal (for example) may be required with no need to axially resolve individual vessels within the retina. In fact, it may be preferable in some implementations to not axially resolve the individual vessels, as the requirements of such axial resolution adds an additional dimension (i.e. depth coordinates) to the recorded image set, thereby dramatically increasing the amount of data that must be stored and processed. The preferred in some circumstances implementation of the proposed LSCI apparatus, therefore, is configured to produce two-dimensional, non-depth resolved images at high frame rates (100 s of frames per second), detecting light reflecting off of the retina, but rejecting light reflecting off of the choroid.

Referring again to FIG. 2, in one implementation, the optical detection system 230, included a Lucid Vision 2k×2k monochrome camera, capable of delivering critically sampled LSCI images with at least a 15 degree FOV (in a related implementation—at least a 30 degree FOV) and 8 micron spatial resolution at 480 fps. In order to reconstruct the coherent image, four LSCI frames are required; thus, this system is able to deliver quantitative flow measurements of blood in vasculature of a portion of the eye, for example, across the entire 15 degree (or 30 degree) FOV at 120 Hz, sufficiently fast to capture retinal hemodynamics in humans.

This novel instrument design allows for accurate axial sectioning of either the retinal or choroidal vasculature with a LSCI modality, allowing hemodynamic alterations to be measured in one or other tissues independently. This has profound implications for the diagnosis and clinical management of diseases including (but not limited to) diabetic retinopathy, age-related macular degeneration (AMD), hypertension and dementia (including Alzheimer's disease), which present with hemodynamic alterations specifically in either the choroidal vasculature (AMD) or retinal microvasculature (other diseases) that cannot currently be addressed using existing imaging modalities.

### Example 2

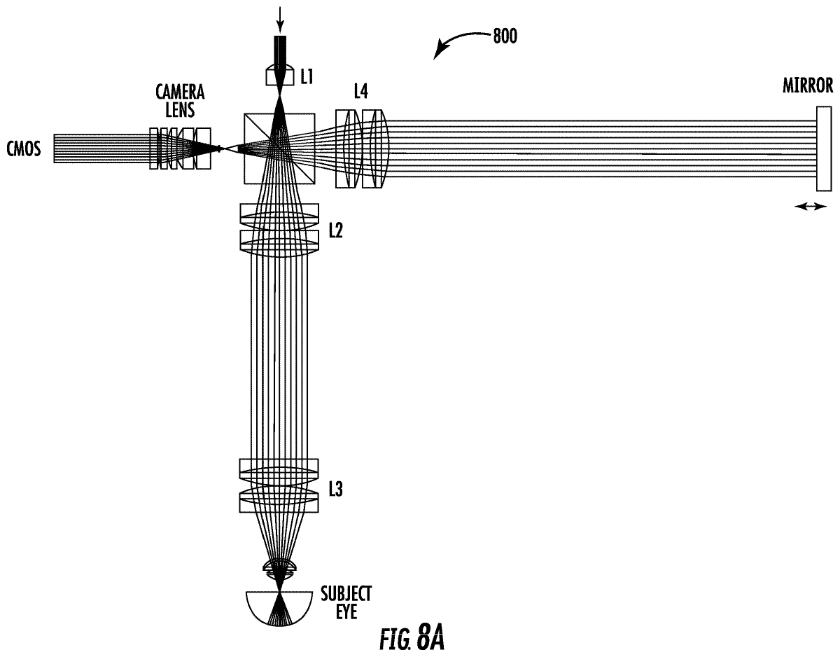

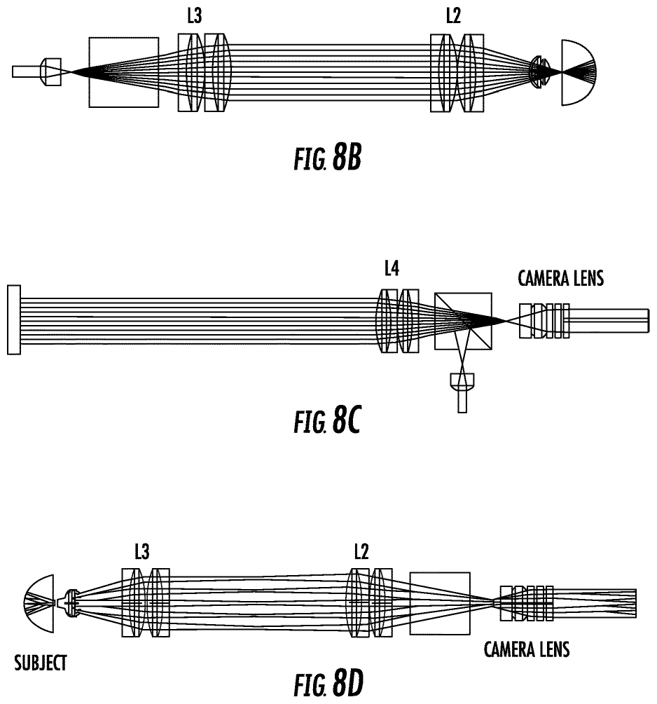

FIG. 8A provides a schematic of a related and operationally comparable with the embodiments discussed above optical imaging apparatus 800 of the system structured according to the idea of the invention. In the embodiment 800, optics of at least the sample arm of the interferometer is somewhat modified as compared with that discussed in Example 1. Additionally, as will be understood from the following discussion, the adjustment of at least temporal coherence of light delivered to the optical interferometer through lens L1 of FIG. 8A from the source of light (along an arrow, as indicated) is configured at the source of light itself—in comparison with the use of the filter F in the case of Example 1. FIGS. 8B, 8C, and 8D complement FIG. 8A by schematically displaying, respectively, portions of the embodiment 800 of the optical imaging apparatus that the skilled artisan will readily identify—a combination of the illumination portion of the apparatus and the sample arm of the constituent interferometer (FIG. 8B), a combination of the illumination portion of the apparatus, the reference arm of the constituent interferometer, and a collections optics marked as a camera lens (FIG. 8C), and a combination of the sample arm of the constituent interferometer and the camera lens (FIG. 8D).

While the complete detailed description of this design of the optical trains of the optical imaging apparatus is not provided here in order to not unnecessarily complicate the disclosure, some relevant data that may inform the skilled person about the details of the design of the combination of optics shown in FIG. 8D is presented below in the Appendix, to provide the required written description and to enable a skilled person to appreciate the specifics of the design of Example 2 in reference to FIG. 8A. Here, some preliminary notes may be in order. In particular, the data presented in Appendix is primarily focused on providing the summary of the of the optical surfaces of the combination of elements in FIG. 8D. The numbering of the surfaces starts with “10” (because surfaces “1” through “9” and “15” through “20” are the surfaces of other portions of the overall optics, FIG. 8A). The notation is substantially standard for Zemax® software, with the use of which this design was carried out. Glass notations are those from the Schott catalog of glasses. Substantially inconsequential for the purposes of this disclosure details—such as, for example, vignetting factors, surface data detail, edge thickness data for constituent lens elements, index of refraction data, global vertex coordinates, orientations, and rotation/offset matrices, etc that Zemax® generates—are not included for the simplicity of presentation.

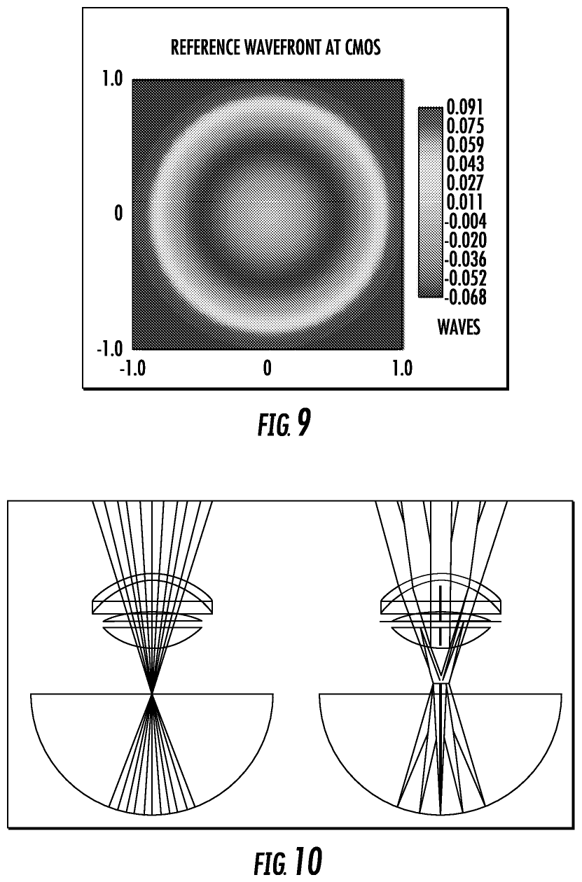

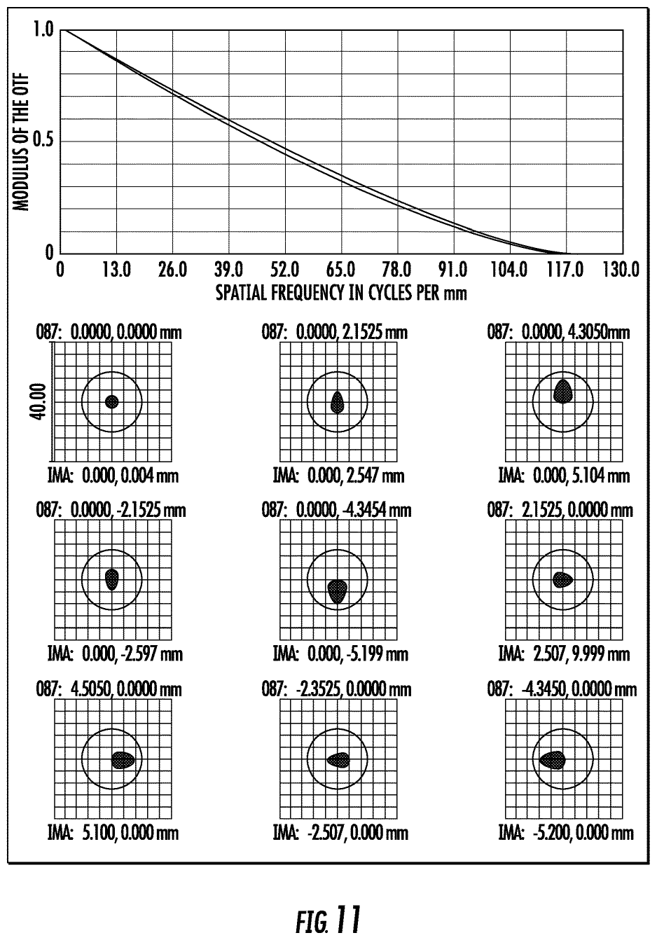

FIG. 9 depicts a modeled reference wavefront at a surface of the CMOS of the specific embodiment of FIG. 8, demonstrating a wavefront error that is smaller than 0.16λpeak-to-valley. FIG. 10 provides an illustration of the path of illumination of the curved layer of the object (in this non-limiting example chosen to be a curved layer of an eye)—on the left, and an illustration of the path of collection of light in reflection from (or scattering by) such curved layer of the object—on the right. Both illustrations shown light beams pivoting through the center of curvature of the curved layer of the object. FIG. 11 presents both the plots representing the modulated transfer function (MTF; top portion of the drawing) and spot diagrams (bottom) depicting diffraction limited performance of the embodiment of FIG. 8A while imaging the retina of an eye to CMOS.

As the person of ordinary skill in the art now readily appreciates, other operational characteristics/parameters of the embodiment 800 are substantially similar or substantially equal to those of the optical imaging apparatus of Example 1.

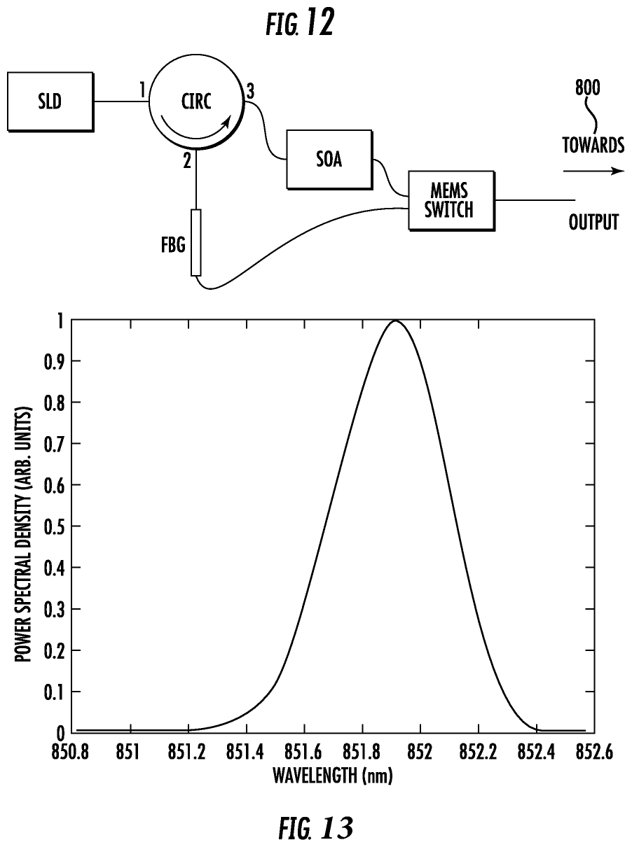

At the same time, the source of light (with which this embodiment of the optical imaging apparatus 800 can be equipped to form a system of the invention) may be unconventionally structured to ensure that light delivered through lens L1 has judiciously engineered coherence. To this end, FIGS. 12 and 13 provide information illustrating an embodiment of such source of light for advantageous use with an embodiment of the optical imaging apparatus of the invention.

As shown in FIG. 12, for example, polarization maintaining (PM) fiber-coupled light emitter—in this non-limiting example represented by a superluminescent diode (SLD) that operates at about 850 nm with a FWHM bandwidth of about 27 nm—directs light from the light emitter towards a PM multi-port optical device (here—represented by an optical circulator, CIRC), which then directs light to an optical reflector through a corresponding port of the multi-port optical device. In one specific case, depicted in FIG. 12, the optical reflector in chosen to be a custom PM fiber Bragg grating (FBG). In this specific case, the FBG has a peak reflectivity of 93% at 851.92 nm and a FWHM of 0.47 nm.

Light reflected from the FBG returns to the circulator and is then directed to a semiconductor optical amplifier (SOA). Both output from the SOA and output from (throughput of) the optical reflector are directed towards an optical switch (here—a MEMS switch), the output of which can then be rapidly switched/changed/chosen from the low temporal coherence output of the optical reflector (equivalent to the SLD output with a notch), or the engineered coherence output of the SOA (see FIG. 13). The SOA output was measured on an optical spectrum analyzer (OSA) to have a FWHM bandwidth of 0.46 nm, nearly identical to that of the optical reflector (here—a FBG). Gain of the SOA was set to stabilize the output power to 20 mW.

It is understood, therefore, that, depending on the specific implementation of the system of the invention, the optical imaging apparatus (containing an optical interferometer as discussed above) may be complemented by any of or any combination of the following:
an optical circulator, an optical reflector, an optical amplifier, and an optical switch (which is optically separated from the optical circulator by each of each of the optical circulator, the optical reflector, and the optical amplifier);
a source of light that includes light emitter optically coupled to a first port of a multi-port optical device, an optical reflector optically coupled to a second port of such multi-port optical device, and an optical switch optically coupled to both the second port and a third port of the multi-port optical device;
a source of light that includes an input defined by a light emitter having a first spectral bandwidth, an intermediate portion of the source of light, and an output defined by an optical switch that is optically separated from the input by said intermediate portion. Here, the intermediate portion is configured to deliver light from the input to the output along first and second optical paths; the first optical path is configured to include an optical reflector therein and to deliver from the light emitter to the output a first light having a bandwidth that is substantially equal to the first bandwidth and the second optical path is configured to deliver from the light emitter to the output a second light having a spectral bandwidth substantially equal to a spectral bandwidth of the optical reflector; and
a source of light is configured to deliver towards the optical interferometer an optical output formed interchangeably in first and second optical output modes, where a spectral bandwidth of the optical output in the first output mode is at least ten times narrower than that in the second output mode.

In at least one case, control of the optomechanical components, image acquisition, and data processing may be performed using a custom software. Accordingly, at least one embodiment may include an electronic circuitry/processor controlled by instructions stored in a memory. The memory may be random access memory (RAM), read-only memory (ROM), flash memory or any other memory, or combination thereof, suitable for storing control software or other instructions and data. Those skilled in the art should also readily appreciate that instructions or programs defining the functions of the present invention may be delivered to a processor in many forms, including, but not limited to, information permanently stored on non-writable storage media (e.g. read-only memory devices within a computer, such as ROM, or devices readable by a computer I/O attachment, such as CD-ROM or DVD disks), information alterably stored on writable storage media (e.g. floppy disks, removable flash memory and hard drives) or information conveyed to a computer through communication media, including wired or wireless computer networks. In addition, while the invention may be embodied in software, the functions necessary to implement the invention may optionally or alternatively be embodied in part or in whole using firmware and/or hardware components, such as combinatorial logic, Application Specific Integrated Circuits (ASICs). Field-Programmable Gate Arrays (FPGAs) or other hardware or some combination of hardware, software and/or firmware components.

While the invention is described through the above-described exemplary embodiments, it will be understood by those of ordinary skill in the art that modifications to, and variations of, the illustrated embodiments may be made without departing from the inventive concepts disclosed herein. For example, in a related the LSCI apparatus of the invention, was configured to image moving elements of the object over a FOV of 30 degrees (in another case=of 15 degrees) in the murine eye. The device also achieved frame rates up to 376 fps over the entire FOV, enabling resolution of pulsatile flow, even at the elevated murine heart rate (up to 840 bps).

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention.

For the purposes of this disclosure and the appended claims, the use of the terms “substantially”, “approximately”, “about” and similar terms in reference to a descriptor of a value, element, property or characteristic at hand is intended to emphasize that the value, element, property, or characteristic referred to, while not necessarily being exactly as stated, would nevertheless be considered, for practical purposes, as stated by a person of skill in the art. These terms, as applied to a specified characteristic or quality descriptor means “mostly”, “mainly”, “considerably”, “by and large”, “essentially”, “to great or significant extent”, “largely but not necessarily wholly the same” such as to reasonably denote language of approximation and describe the specified characteristic or descriptor so that its scope would be understood by a person of ordinary skill in the art. In one specific case, the terms “approximately”, “substantially”, and “about”, when used in reference to a numerical value, represent a range of plus or minus 20% with respect to the specified value, more preferably plus or minus 10%, even more preferably plus or minus 5%, most preferably plus or minus 2% with respect to the specified value. As a non-limiting example, two values being “substantially equal” to one another implies that the difference between the two values may be within the range of +/−20% of the value itself, preferably within the +/−10% range of the value itself, more preferably within the range of +/−5% of the value itself, and even more preferably within the range of +/−2% or less of the value itself. The use of these terms in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated falls and may vary within a numerical range defined by an experimental measurement error that is typical when using a measurement method accepted in the art for such purposes.

The use of these terms in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated falls and may vary within a numerical range defined by an experimental measurement error that is typical when using a measurement method accepted in the art for such purposes.

The term “A and/or B” or a similar term means “A alone, B alone, or A and B together” and is defined to be interchangeable with the term “at least one of A and B.”

The term “image” refers to and is defined as an ordered representation of detector signals corresponding to spatial positions. For example, an image may be an array of values within an electronic memory, or, alternatively, a visual or visually-perceivable image may be formed on a display device such as a video screen or printer.

A real-time performance of a system is understood and defined as performance which is subject to operational deadlines from a given event to a system's response to that event. For example, a real-time extraction of optical information (such as irradiance distribution across an image formed with the optical imaging apparatus from the optical detector or sensor disposed at a surface characterized by such irradiance distribution) may be one triggered by the user or a processor and executed simultaneously with and without interruption of the optical imaging process during which such image has been acquired.

While the invention is described through the above-described exemplary embodiments, it will be understood by those of ordinary skill in the art that modifications to, and variations of, the illustrated embodiments may be made without departing from the inventive concepts disclosed herein. Disclosed aspects, or portions of these aspects, may be combined in ways not listed above. Accordingly, the invention should not be viewed as being limited to the disclosed embodiment(s).

APPENDIX
Some System/Prescription Data
GENERAL LENS DATA:

| | |
|---|---|
| Surfaces | 60 |
| Stop | 45 |
| System Aperture | Float By Stop Size = 1.05 |
| Fast Semi-Diameters | On |
| Field Unpolarized | On |
| Convert thin film phase | On |
| to ray equivalent | |
| J/E Conversion Method | X Axis Reference |
| Glass Catalogs | CDGM SCHOTT EYE_JAEKEN_THEIA |
| | EYE_JAEKEN AZEYE15 |
| Ray Aiming | Real Reference, Cache On |
| X Pupil Shift | 0 |
| Y Pupil Shift | 0 |
| Z Pupil Shift | 0 |
| X Pupil Compress | 0 |
| Y Pupil Compress | 0 |
| Apodization | Uniform, factor = 1.00000E+00 |
| Reference OPD | Exit Pupil |
| Paraxial Rays Setting | Ignore Coordinate Breaks |
| Method to Compute F/# | Tracing Rays |
| Method to Compute | Force Planar |
| Huygens Integral | |
| Print Coordinate Breaks | On |
| Multi-Threading | On |
| OPD Modulo 2 Pi | Off |
| Temperature (C.) | 2.00000E+01 |
| Pressure (ATM) | 1.00000E+00 |
| Adjust Index Data | Off |
| To Environment | |
| Effective Focal Length | −10.18604 (in air at system |
| | temperature and pressure) |
| Effective Focal Length | −10.18604 (in image space) |
| Back Focal Length | 23.20545 |
| Total Track | 237.8965 |
| Image Space F/# | 9.040194 |
| Paraxial Working F/# | 9.644123 |
| Working F/# | 9.637279 |
| Image Space NA | 0.05177551 |
| Object Space NA | 0.06010323 |
| Stop Radius | −1.05 |
| Paraxial Image Height | 5.014815 |
| Paraxial Magnification | 1.164882 |
| Entrance Pupil Diameter | 1.12675 |
| Entrance Pupil Position | −4.19503 |
| Exit Pupil Diameter | 19.84813 |
| Exit Pupil Position | −191.3062 |
| Field Type | Object height in Millimeters |
| Maximum Radial Field | 4.305 |
| Primary Wavelength [μm] | 0.8519 |
| Angular Magnification | −0.0756396 |
| Lens Units | Millimeters |
| Source Units | Watts |
| Analysis Units | Watts/cm\hat{ }2 |
| Afocal Mode Units | milliradians |
| MTF Units | cycles/millimeter |
| Include Calculated | On |
| Data in Session File | |
| Include Calculated | On |
| Data in Session File | |

Fields: 9
Field Type: Object height in Millimeters

| # | X-Value | Y-Value | Weight |
|---|---|---|---|
| 1 | 0.000000 | 0.000000 | 1.000000 |
| 2 | 0.000000 | 2.152500 | 1.000000 |
| 3 | 0.000000 | 4.305000 | 1.000000 |
| 4 | 0.000000 | −2.152500 | 1.000000 |
| 5 | 0.000000 | −4.305000 | 1.000000 |
| 6 | 2.152500 | 0.000000 | 1.000000 |
| 7 | 4.305000 | 0.000000 | 1.000000 |
| 8 | −2.152500 | 0.000000 | 1.000000 |
| 9 | −4.305000 | 0.000000 | 1.000000 |

Wavelengths: 3
Units: μm

| # | Value | Weight |
|---|---|---|
| 1 | 0.851700 | 1.000000 |
| 2 | 0.851900 | 1.000000 |
| 3 | 0.852100 | 1.000000 |

Predicted coordinate ABCD matrix:

| | |
|---|---|
| A= | 1.156 |
| B= | 0.002776 |
| C= | 0.002776 |
| D= | 1.156 |

### Surface Data Summary:

SURFACE DATA SUMMARY:

| Surf | Type | Radius | Thickness | Glass | Clear Diam | Chip Zone | Mech Diam | Conic | Comment |
|---|---|---|---|---|---|---|---|---|---|
| OBJ | STRD | 12 | 16.64 | VITREOUS_JAEK | 24 | 0 | 24 | 0 | Goncharov Retina |
| 10 | GRINSUR5 | 7.67 | 3.69 | GLENS_JAEK | 10 | 0 | 24 | 0.5 | Posterior Lens |
| 11 | STRD | −11.51 | 0 | AQUEOUS_JAEK | 10 | 0 | 12 | −1 | Anterior Lens |
| 12 | STRD | −11.51 | 3.06 | AQUEOUS_JAEK | 2 | 0 | 12 | 0 | Pupil |
| 13 | STRD | −6.52 | 0.55 | CORNEA_JAEK | 12 | 0 | 12 | −0.3 | Post Cornea |
| 14 | STRD | −7.76 | 33.52 | | 12 | 0 | 12 | −0.1 | Anterior Cornea |
| 21 | STRD | Inf | −16.386 | | 26.928 | 0 | 26.928 | 0 | |
| 22 | STRD | Inf | 0 | | 18.079 | 0 | 18.079 | 0 | ADJUST |
| 23 | COORDBRK | 0 | | | — | — | — | — | |
| 24 | STRD | −467.36 | 3 | H-ZF7LA | 24 | 1.5 | 28 | 0 | D1_S1 |
| 25 | STRD | 57.24 | 6.5 | H-LAK10 | 25.2 | 1.4 | 28 | 0 | D1_S2 |
| 26 | STRD | −39.21 | 0.1 | | 25.2 | 1.4 | 28 | 0 | D1_S3 |
| 27 | STRD | 39.21 | 6.5 | H-LAK10 | 25.2 | 1.4 | 28 | 0 | D2_S1 |
| 28 | STRD | −57.24 | 3 | H-ZF7LA | 25.2 | 1.4 | 28 | 0 | D2_S2 |
| 29 | STRD | 467.36 | 33.528 | | 24 | 1.5 | 28 | 0 | D2_S3 |
| 30 | STRD | Inf | 0 | | 20.411 | 0 | 20.411 | 0 | |
| 31 | STRD | Inf | 0.004354 | | 20.41106 | 0 | 20.411 | 0 | FOCUS ADJUST |
| 32 | STRD | Inf | 39.92464 | | 20.4114 | 0 | 20.411 | 0 | |
| 33 | STRD | 39.21 | 6.5 | H-LAK10 | 25.2 | 1.4 | 28 | 0 | D3_S1 |
| 34 | STRD | −57.24 | 3 | H-ZF7LA | 25.2 | 1.4 | 28 | 0 | D3_S2 |
| 35 | STRD | 467.36 | 0.1 | | 24 | 1.5 | 28 | 0 | D3_S3 |
| 36 | STRD | 39.21 | 6.5 | H-LAK10 | 25.2 | 1.4 | 28 | 0 | D4_S1 |
| 37 | STRD | −57.24 | 3 | H-ZF7LA | 25.2 | 1.4 | 28 | 0 | D4_S2 |
| 38 | STRD | 467.36 | 7.5 | | 24 | 1.5 | 28 | 0 | D4_S3 |
| 39 | STRD | Inf | 25 | N-BK7 | 20.3 | 0 | 20.3 | 0 | BS_S1 |
| 40 | STRD | Inf | 7.022 | | 20.3 | 0 | 20.3 | 0 | BS_S2 |
| 41 | STRD | Inf | 0.26 | | 3.345 | 0 | 3.345 | 0 | |
| 42 | STRD | Inf | 4.94 | | 3.210 | 0 | 3.210 | 0 | |
| 43 | STRD | Inf | 0.7 | | 3.5276 | 0 | 3.528 | 0 | |
| 44 | STRD | Inf | −3.51 | | 3.883 | 0 | 3.883 | 0 | ADJUST |
| STO | STRD | Inf | 0 | | 2.1 | 0 | 2.1 | 0 | |
| 46 | STRD | Inf | 3.51 | | 2.1 | 0 | 2.1 | 0 | |
| 47 | STRD | −9.08 | 5.5 | H-ZF7LA | 4.4 | 0.3 | 12 | 0 | E1_S1 |
| 48 | STRD | −37.41 | 0.86 | | 6.9 | 4.05 | 15 | 0 | E1_S2 |
| 49 | STRD | −16.07 | 5.5 | H-ZF7LA | 7.4 | 0.55 | 15 | 0 | E2_S1 |
| 50 | STRD | −14.05 | 0.2 | | 10.6 | 2.2 | 15 | 0 | E2_S2 |
| 51 | STRD | −46.88 | 3 | H-ZF7LA | 11 | 0.7 | 15 | 0 | E3_S1 |
| 52 | STRD | −24.72 | 0.2 | | 12 | 1.5 | 15 | 0 | E3_S2 |
| 53 | STRD | Inf | 3 | H-ZF7LA | 13.2 | 0.9 | 15 | 0 | E4_S1 |
| 54 | STRD | −56.16 | 0.2 | | 13.2 | 0.9 | 15 | 0 | E4_S2 |
| 55 | STRD | 56.16 | 3 | H-ZF7LA | 13.2 | 0.9 | 15 | 0 | E5_S1 |
| 56 | STRD | Inf | 0 | | 13.2 | 0.9 | 15 | 0 | E5_S2 |
| 57 | STRD | Inf | 0 | | 11.881 | 0 | 11.881 | 0 | |
| 58 | STRD | Inf | 34.916 | | 11.881 | 0 | 11.881 | 0 | |
| 59 | STRD | Inf | 0 | | 10.215 | 0 | 10.215 | 0 | |
| IMA | STRD | Inf | | | 10.215 | 0 | 10.21457 | 0 | |

What is claimed is:

1. A system comprising an optical imaging apparatus that includes:
an optical interferometer having a sample arm and a reference arm; and
an optical relay system in the sample arm, the optical relay system configured a) to include first and second lenses axially separated by a distance exceeding a sum of focal lengths of said first and second lenses, and b) to form a spatially-curved image surface having a curvature that is necessarily variable as a function of said distance and/or a working distance of the optical imaging apparatus.

2. (canceled)

3. (canceled)

4. A system according to claim 1, wherein the first and second lenses are substantially identical optical doublets.

5. A system according to claim 1, configured as an ophthalmoscope and further comprising a source of light apparatus having coherence length that is necessarily substantially within a range from about 200 microns to about 500 nm.

6. A system according to claim 1, comprising a source of light and configured to irradiate an object with light formed at an output of said source of light while registering, at an optical detector of the system, a laser speckle representing said object.

7. A system according to claim 6, wherein said source of light is configured to interchangeably form said light either as a first light or as a second light, the first and second light having corresponding degrees of temporal coherence that are different from one another and/or corresponding degrees of spatial coherence that are different from one another.

8. A system according to claim 1, comprising a source of light that includes light emitter optically coupled to a first port of a multi-port optical device, an optical reflector optically coupled to a second port of said multi-port optical device, and an optical switch optically coupled to both the second port and a third port of the multi-port optical device.

9. A system according to claim 1,
comprising a source of light that includes an input defined by a light emitter having a first spectral bandwidth, an intermediate portion of the source of light, and an output defined by an optical switch that is optically separated from the input by said intermediate portion,
wherein the intermediate portion is configured to deliver light from the input to the output along first and second optical paths,
wherein the first optical path is configured to include an optical reflector therein and to deliver from the light emitter to the output a first light having a bandwidth that is substantially equal to the first bandwidth while the second optical path is configured to deliver from the light emitter to the output a second light having a spectral bandwidth substantially equal to a spectral bandwidth of the optical reflector.

10. A system according to claim 6, wherein the source of light is configured to deliver towards the optical interferometer an optical output formed interchangeably in first and second optical output modes, wherein a spectral bandwidth of the optical output in the first output mode is at least ten times narrower than that in the second output mode.

11. A system according to claim 1, further comprising an optical circulator, an optical reflector, an optical amplifier, and an optical switch that is optically separated from the optical circulator by each of each of the optical circulator, the optical reflector, and the optical amplifier.

12. A system according to claim 1, further comprising a programmable data-processing electronic circuitry, operably connected with an optical detector of the system and tangible non-transitory storage medium, wherein said storage medium contains program code thereon, the program code configured to determine an index of motion at an object irradiate with light that has traversed the optical interferometer based at least in part on a speckle contrast characteristic of at least one optical image of the object formed at the optical detector.

13. A system according to claim 12, wherein the object includes a layer of retina or a layer of choroid, and wherein the program code is configured to determine a blood flow rate in a reference blood-vessel of the object said layer of retina or said layer of choroid.

14. A method comprising:
with the use of the system according to claim 1:
dynamically matching a first spatial curvature of an arcuate surface of illumination, formed by illuminating light propagating from a source of light of the system through an illumination sub-system with a second spatial curvature of a spatially curved object layer of a material, wherein the illumination sub-system includes a sample arm of an optical interferometer;
irradiating the spatially curved object layer of a material with the illuminating light by substantially spatially superimposing said arcuate surface of illumination with said spatially curved object layer of a material;
and
at an optical detector of a light collecting sub-system, forming an optical image of said curved object layer of a material by spatially overlapping sample light, which has been returned from said spatially curved object layer irradiated with the illuminating light, with a portion of the illuminating light that has been delayed with respect to the sample light.

15. A method according to claim 14,
(A) wherein said dynamically matching is carried out substantially simultaneously with said irradiating, and/or
(B) wherein said illuminating surface is substantially optically-conjugate with the source of light.

16. A method according to claim 1, wherein said irradiating includes irradiating the spatially curved object layer of a material with the illuminating light having a coherence length that necessarily does not exceed a thickness of said spatially curved object layer of a material and/or that is necessarily substantially equal to said thickness.

17. A method according to claim 16, wherein said spatially curved object layer of a material includes a spatially curved stack of multiple material layers.

18. A method according to claim 14, wherein said dynamically matching includes axially repositioning a first lens of a multi-lens optical relay system that is contained within the sample arm.

19. A method according to claim 14, wherein said forming an optical image includes forming an interferometric image of only the spatially curved object layer of a material and not of another portion of an object space adjacent to the spatially curved object layer of a material by optically interfering the sample light with the portion of illuminating light at the optical detector.

20. A method according to claim 14, further comprising generating first and second images of the spatially curved object layer of a material respectively representing said curved layer at different depths thereof by changing a delay between the sample light and the portion of illuminating light.

21. A method according to claim 20, wherein the spatially curved object layer of a material includes only one structural layer of multiple structural layers located at a back of an eye, and wherein said generating includes depth-wise imaging of said only one structural layer while not imaging another of said multiple structural layers.

22. A method according to claim 14, wherein:
(a) said substantially matching includes substantially matching the first spatial curvature with the second spatial curvature over at least a 15-degree full field of view (FOV) of the illumination sub-system, and/or over at least a 20-degree full FOV of the illumination system, and/or over at least a 30-degree full FOV of the illumination sub-system; and/or
(b) said forming an optical image includes forming an optical image simultaneously representing a microvasculature of the retina of an eve over at least a 15-degree full FOV, and/or over at least a 20-degree full FOV, and/or over at least a 30-degree full FOV.

23. (canceled)

24. A method according to claim 14, comprising:
forming said illuminating light interchangeably as a first illuminating light or as a second illuminating light by transmitting light, that has been emitted by a light emitter, through an optical switch that optically separates the light emitter from the optical interferometer, wherein the first and second illuminating lights have corresponding first and second spectral bandwidths that are different from one another by at least an order of magnitude.

25. A method according to claim 24, comprising:
forming said second illuminating light at least in part by spatially overlapping (i) a first portion of the light that has been emitted by the light emitter and that has been reflected at an optical reflector and (ii) a second portion of the light that has been emitted by the light emitter.

26. A method according to claim 25, further comprising amplifying both the first portion and the second portion that have been spatially overlapped.

27. A method according to claim 25, wherein said spatially overlapping includes transmitting both the first portion and the second portion through the same port of a multi-port optical device that is optically separated from the optical switch by said optical reflector.

28. A method according to claim 24, comprising delivering to light that has been emitted by the light emitter to the optical switch along first and second spatially distinct optical paths, the first optical path traversing an optical circulator and a fiber Bragg grating and the second optical path traversing the optical circulator and an optical amplifier.

29. A method according to claim 14, further comprising:
with the use of a programmable data-processing electronic circuitry, operably connected with the optical detector, determining an index of motion occurring in the spatially curved object layer based at least in part on a speckle contrast characteristic of said optical image of the spatially curved object layer.

30. A method according to claim 29, further comprising generating a visually-perceivable output representing changes in dynamics of said motion over a predetermined period of time based at least on said determining.

31. (canceled)

32. A method according to claim 29,
wherein the curved object layer of material is a layer of retina or a layer of choroid, and
wherein said determining an index of motion includes determining a blood flow rate in a reference blood-vessel curved object layer of material from at least said optical image of the curved object layer of material.

33. A method according to claim 32, further comprising generating a visually-perceivable output representing quantified changes in retinal hemodynamics over a predetermined period of time based at least on said determining.
